(19) United States
(12) Patent Application Publication
(10) Pub. No.: US 2011/0097367 A1
(43) Pub. Date: Apr. 28, 2011

(54) MONOLITHIC IN-SITU CROSS-LINKED ALGINATE IMPLANTS

(75) Inventors: Christine Wallrapp, Grossostheim (DE); Herma Glöckner, Kleinwallstadt (DE); Roland Reiner, Darmstadt (DE); Frank Thürmer, Alzenau (DE)

(73) Assignee: CELLMED AG

(21) Appl. No.: 12/863,387

(22) PCT Filed: Jan. 9, 2009
(86) PCT No.: PCT/EP2009/000094

§ 371 Date: Jan. 4, 2011

(30) Foreign Application Priority Data
Jan. 16, 2008 (EP) 08000745.3

(51) Int. Cl. A61K 31/734; A61K 35/00; A61K 38/00; A61K 38/27; A61K 38/25; A61K 38/19; A61K 38/21; A61K 38/20; A61K 38/18; A61K 38/28; A61K 38/30; A61K 39/395; A61K 38/49; A61K 8/02; A61K 8/73; A61K 8/64; A61K 8/96; A61K 9/00; A61Q 19/08; A61P 1/04; A61P 13/00; A61P 35/00; A61P 41/00

(52) U.S. Cl. 424/401; 514/54; 424/93.1; 514/1.1; 514/11.4; 514/11.3; 514/11.2; 424/85.1; 424/85.4; 424/85.2; 514/7.7; 514/8.8; 514/6.5; 514/8.5; 514/9.6; 424/141.1; 424/94.64; 424/423

(57) ABSTRACT

A method of making and using a monolithic alginate implant is described. The implant is formed by providing an uncrosslinked, highly pure and high molecular weight alginate solution and injecting the alginate solution into a patient at a predetermined site to form a gel body comprising the monolithic alginate implant. Spontaneous crosslinking of the monolithic alginate implant occurs at the predetermined site without the addition of an exogenous crosslinker. The implant may be used for treating medical conditions requiring support of sphincter musculature, reconstructive surgery, or cosmetic reconstruction, for the treatment of wrinkles on the hand, face, or décolleté, or for increasing volume, for example in the case of (HIV-induced) lipoatrophy of the breasts, and for the treatment of selected diseases, including gastrooesophageal reflux disease, urinary incontinence or vesicoureteral reflux disease.

### BACKGROUND OF THE INVENTION

In the field of medicine and cosmetics, a large demand for filler materials has arisen, in particular in recent years, in order advantageously to support skin and muscle properties due to aesthetic, disease- or age-related circumstances by volume enhancement. Such aesthetic, disease- or age-related circumstances concern inter alia the formation of wrinkles. Wrinkles form even during childhood as a result of facial expressions, in later life typically as a result of physical damage such as the sun, heat, environment, and during old age as a result of normal skin ageing. Wrinkles can also be caused by illnesses which lead to the displacement of the fatty tissue in the body, for example fatty tissue atrophy with regression of fatty tissue accompanied by the formation of wrinkles in the dermal layer covering the tissue, such as, for example, in the case of lipoatrophy, in particular HIV-induced lipoatrophy, which leads to considerable atrophy of the fatty tissue especially in the region of the extremities and the cheeks.

In order to comply with many patients' continuous desire for a youthful appearance, various methods of treating wrinkles and increasing the volume of the skin or the tissue lying beneath it have in the meantime become established.

On the one hand, so-called chemical denervation or inactivation of the muscles or muscle groups in question can be carried out. There are used in this connection in particular compounds that are able to block the release of a messenger substance for triggering muscle contractions, such as, for example, botulinum toxin A or derivatives thereof. Known preparations are marketed, for example, under the names Botox®, Dysport®, Vistabel® or Xeomin®. These compounds bring about, typically in the region of the treated musculature, an inactivation of the injected muscles or muscle groups, the smoothing effect lasting from a few months to a year depending on the activity of the musculature. Such methods therefore contribute towards the desired smoothing of the skin only when used regularly.

Other methods of wrinkle treatment which have been developed are based on leveling the surface of the skin to be treated by treatment with so-called “fillers”. In the case of treatment with “fillers”, the dermis is lined with endogenous or exogenous substances and the tissue is thereby stabilised and, optionally, smoothed. In Europe, a large number of exogenous fillers are available, which consist predominantly of biological substances such as collagen or hyaluronic acid (e.g. of collagen: Zyderm®, Zyplast®, Atelocollagen®, Resoplast®; of hyaluronic acid: Hylaform®, Restylane®, Belotero®, Perlane®, Juvederm®, Rofilan Hylan Gel®, Hyal-System®, Viscontur®).

In this context, collagen is a natural protein which occurs both in humans and in animals and which keeps (human) connective tissue elastic. Collagen preparations for injection are typically obtained from porcine or bovine collagen. When porcine and bovine collagen is used, however, allergic reactions to these protein products can occur in humans, so that it is necessary to carry out allergy tests before use. Another disadvantage of collagen preparations is that fact that collagen can migrate from the injection site to different areas of the skin, possibly causing redness and swelling there (Millikan, 1989, Long term safety and Efficacy with Fibrel in the treatment of cutaneous scars, J Dermatol Surg Oncol, 15:837-842).

Hyaluronic acid, which can likewise be present in exogenous fillers, is a mucopolysaccharide which occurs in almost every part of a living organism and in particular in the skin Chemically, hyaluronic acid is formed of straight polymer chains having a molecular weight in the range of from several hundred thousand to millions of Daltons, which chains contain repeating disaccharide units of N-acetylglucosamine and glucuronic acid bonded together by glycosidic bonds. A comparative study to evaluate the clinical usability of collagen preparations on the one hand and hyaluronic acid preparations on the other showed that the hyaluronic acid-based product Restylane® yields markedly better results than the collagen preparation Zyderm® (Narins et al., 2003, A randomized, double-blind, multicenter comparison of the efficacy and tolerability of restylane versus zyplast for the correction of nasolabial folds. Dermatol. Surg., 29: 588-95). However, a disadvantage of hyaluronic acid preparations is that, in order to achieve a visible effect, the skin must be treated up to three times at short intervals. This can lead to swellings, which only subside after 1 to 2 days and therefore makes treatment very complex. In addition, both hyaluronic acid and collagenic acid preparations are known to have complications two to three years post-injection—at a time at which the injected materials have long been degraded (Hanke et al., 1991, Abscess formation and local necrosis after treatment with Zyderm or Zyplast Collagen Implant. Journal of American Academy of Dermatology, 25 (No. 2, Part 1): 319-26; Moscona et al., 1993, An unusual late reaction to Zyderm I injections: A challenge for treatment. Plastic and reconstructive surgery, 92: 331-4). Finally, the human (and animal) body produces enzymes which degrade hyaluronic acid. Treatment solely with hyaluronic acid or with preparations that contain substantially hyaluronic acid or hyaluronic acid as a major constituent therefore leads to a rapid disappearance of the visible effect and typically requires the treatment to be repeated comparatively frequently. Nowadays, hyaluronic acid is often used in the form of crosslinked hyaluronic acid. Although the effect of the degradation of natural hyaluronic acid in the tissue and the rapid disappearance of the visible effect of increased volume is thereby reduced, the crosslinked hyaluronic acid compounds that are used are typically hyaluronic acids which have been massively chemically modified and no longer constitute “natural fillers”.

As an alternative to the use of above-mentioned exogenous fillers such as collagen or hyaluronic acid for wrinkle injection, liquid silicone has also been used for a long time. However, numerous disadvantageous side-effects have also been found in this connection, such as, for example, the formation of nodules, periodically recurring cellulitis and the formation of skin ulcers. Treatment with silicone is therefore no longer recommended (e.g. Edgerton et al., 1976, Indications for and pitfalls of soft tissue augmentation with liquid silicone, Plast. Reconstr. Surg. 58: 157-65).

As a further alternative to exogenous fillers, implants based on crosslinked alginates have very recently been developed for wrinkle injection in the skin, and their use in the treatment of wrinkles has been successfully tested (see WO 2005/105167, Cellmed AG, Germany). It has thereby been possible especially to prevent the problems mentioned above, for example the necessity of multiple treatment in order to achieve a visible effect, the occurrence of swellings which only subside 1 to 2 days after the injection or implantation. For example, published international patent application WO 2005/105167 (Cellmed AG, Germany) describes implants based on crosslinked alginates for wrinkle injection in the skin which, owing to the low immunogenicity of the alginates, ensure substantially better tolerability of the injected filler material. WO 2005/105167 describes in particular the use of crosslinked alginates in the form of implantable microcapsules or microparticles or of gels of alginates crosslinked with di- or poly-valent cations for use as a “filler” substance and in the treatment of skin deficits, such as, for example, wrinkles. Such implantable microcapsules or microparticles in particular do not result in allergic reactions or an endogenous immune response of the patient. Regardless of their advantages, if the alginate is to be crosslinked in situ it is necessary to administer a crosslinker in parallel, which requires a double cannula and accordingly comparatively complex preparation for the administration. It would therefore be preferred, in the field of wrinkle injection, to provide substances and materials which do not require such an outlay and which can also be injected in relatively large volumes if necessary.

Such endogenous or exogenous filler materials as described above are not only known from cosmetic applications, however, but can also be used to treat selected diseases by injection into the corresponding sphincter musculature, such as, for example, gastrooesophageal reflux disease, urinary incontinence or vesicoureteral reflux disease.

Although gastrooesophageal reflux disease (“GORD”) (gastroesophageal reflux disease (“GERD”)) is a normal physiological phenomenon, it can lead to severe pathophysiological symptoms. Gastrooesophageal reflux disease describes the reflux of acid, enzymatic liquid from the stomach into the oesophagus. It causes heartburn, eructation and regurgitation of the stomach acid into the oral cavity or even into the lungs. The consequences of gastrooesophageal reflux disease are burning of the oesophagus and the formation of ulcers, normal epithelial tissue being replaced by pathological tissue. In healthy patients, the lower oesophageal sphincter muscle closes after food has been ingested. In patients suffering from gastrooesophageal reflux disease, this does not happen. Instead, the muscle typically relaxes and the stomach acid is able to flow into the oesophagus when the stomach contracts. As well as this principal cause of “GORD”, other causes are also possible and known.

Gastrooesophageal reflux disease (“GORD”) is widespread. Statistical data show that about 35% of the American population suffers from heartburn at least once a month and, of these, about 5 to 10% suffer once a day. Medically confirmed endoscopy studies show that 2% of the American population suffers from “GORD”. The risk of suffering from “GORD” increases from the age of 40 (Nebel et al., 1976, Symptomatic gastroesophageal reflux: incidence and precipitating factors, Am. J. Dig. Dis., 21: 953-6). The first signs of gastrooesophageal reflux disease are usually redness which is visible by endoscopy. An advanced stage of the disease can be recognised by destruction of the tissue, followed by neoplasm formation and carcinoma (adenocarcinoma of the oesophagus). Diffuse neoplasm formation occurs in 3.5% of patients below the age of 65 and in 20 to 30% of patients above the age of 65 (Reynolds, 1996, Influence of pathophysiology, severity, and cost on the medical management of gastroesophageal reflux disease. Am. J. Health-Syst. Pharm 53:5-12).

At present, “GORD” is generally treated with proton pump inhibitors, by means of which the majority of patients can be treated successfully with an adequate dosage. However, they have the disadvantage that, owing to the high incidence of recurrence after the acid-suppressing therapy has been stopped, long-term therapy with medicaments is necessary in most patients if conservative long-term elimination of the symptoms is to be achieved (Bittinger and Messmann, 2003, Neue endoskopische Therapieverfahren bei gastroösophagealer Refluxkrankheit, Z. Gastroenerol 41: 921-8). Moreover, many patients are not prepared to take medicaments daily for decades to come. There is the additional problem of the not inconsiderable costs of such long-term therapy with medicaments.

In addition to open and laparoscopic fundoplication, endoscopic therapy methods have recently also been used with the aim of approaching the main cause of gastrooesophageal reflux disease, namely an incompetent lower oesophageal sphincter, therapeutically. Three different basic principles are mostly followed. On the one hand, suture techniques (e.g. endoscopic gastroplasty, full wall oplication) can be used. A radiofrequency application is further possible and, thirdly, injection and implantation methods (e.g. injection of endogenous or exogenous filler materials, biopolymer injection, or implantation therapy) can be used.

Such injection and implantation methods include inter alia also the injection of endogenous or exogenous filler materials. Attempts at supporting the sphincter musculature by injecting swellable substances as natural conventional filler materials, for example the use of bovine collagen or Teflon paste, unfortunately failed, however, because the material migrated from the original injection site over time or was resorbed and accordingly did not permit lasting treatment.

A further alternative to such injection and implantation methods (e.g. biopolymer injection, implantation therapy) includes inter alia the use of ethylene-vinyl alcohol polymer. A corresponding method is currently being carried out using an ethylene-vinyl alcohol polymer (Enteryx®, Boston Scientific, USA). This is a synthetic polymer which is not biodegradable, is chemically inert, does not have antigenic properties and has a permanently spongy/elastic consistency after precipitation in the tissue. After the substance has been dissolved in a solvent (dimethyl sulfoxide), the polymer is injected in the liquid state specifically into the oesophageal wall via an endoscopic injection cannula under radiological control (support for the musculature, raising of the pressure). Within the context of a clinical study, however, it was found to be a disadvantage that in only 60% of patients was more than 50% of the injected polymer still located in situ at the injection site after 6 months, and in some cases more than 75% of the originally injected amount was no longer detectable (Devière et al., 2002, Endoscopic implantation of a biopolymer in the lower esophageal sphincter for gastro-esophageal reflux: a pilot study. Gastrointes Endsc 2002, 55: 335-41). It is clear, therefore, that, in a considerable proportion of patients, the polymer migrates over time, presumably through the wall into the lumen of the gastrointestinal tract. Moreover, the use of dimethyl sulfoxide as solvent for the ethylene-vinyl alcohol polymer used is to be categorised as critical for health reasons. Although biopolymer therapy continues to appear attractive, despite these results, because of the technically comparatively simple methodology and the results obtained hitherto, the irreversibility of the method (synthetic, non-degradable polymer) and the migration of the injected material must be regarded as disadvantageously critical.

An alternative to the injection and implantation methods described above, in particular to biopolymer injection, is based on the use of alginates. WO 2005/105167, for example, describes the use of crosslinked alginates in the form of implantable microcapsules or microparticles or of gels of alginates crosslinked with di- or poly-valent cations for use as a “filler” substance and in the treatment of gastrooesophageal reflux disease. Here too, such implantable microcapsules or microparticles do not exhibit any allergic reactions or the generation of an endogenous immune response of the patient. However, as described hereinbefore for wrinkle therapy, the microcapsules or microparticles disclosed in WO 2005/105167 on the one hand must be prepared prior to administration in a separate preparation process and on the other hand require a not inconsiderable technical outlay during administration. In the field of gastrooesophageal reflux disease too, it would therefore be preferred to provide substances and materials which permit simpler preparation and handling.

A further disease which is of interest in this connection is urinary incontinence, which has already been mentioned above. Urinary incontinence, in which there is an involuntary discharge of urine, can occur as an independent disease or as an accessory symptom to other diseases. Urinary incontinence, which affects more than 6 million people in Germany, is frequently regarded as a taboo subject and is therefore hidden and medical help is scarcely sought. It is therefore difficult to draw up precise figures relating to the occurrence of urinary incontinence. Estimates suggest, however, that in Germany about 11% of people over 65 and 30% of those over 80 are affected by urinary incontinence. Younger people suffering from urinary incontinence are mostly women. The reason for this is that many women have a weakened pelvic floor musculature following pregnancy and childbirth and often attach too little importance to exercising of the pelvic floor after delivery. In later life, urinary incontinence frequently occurs in men as a result of benign prostatic hyperplasia. In addition to the social strain, patients with urinary incontinence are predisposed to urinary tract infections, ulcers, rashes and urinary sepsis. In the USA alone, more than 10 thousand million US dollars are spent every year in dealing with urinary incontinence.

The causes of urinary incontinence can be varied. One cause is weakness of the internal sphincter muscle (M. sphincter urethrae internus) of the bladder musculature. In a particular form of urinary incontinence, vesicoureteral reflux disease, which frequently occurs in younger children, a further cause is reflux of urine through the urethra from the bladder towards the kidneys during urination. Urine reflux can permanently damage the kidneys through bacterial contamination, from scarring to the loss of one or both kidneys. The method of avoiding kidney damage must therefore in that case be the avoidance of kidney infections. Although vesicoureteral reflux in children passes by itself in time, it leads in some cases to severe urinary tract and kidney infections and even to kidney failure.

One form of therapy for such diseases is based on conventional treatment strategies with medicaments. For example, substances having an anticholinergic action that relax the musculature of the urinary bladder are conventionally extensively administered to treat urinary incontinence, in particular when weakness of the internal sphincter muscle has occurred (e.g. Wein, 1995, Pharmacology of Incontinence, Urol. Clin. North Am., 22: 557-77). However, the significant side-effects of such medicaments are often a disadvantage. Furthermore, vesicoureteral reflux disease, the form of urinary incontinence that occurs especially in children, can be treated by the long-term prophylactic administration of antibiotics. In this case, too, however, such treatments are unfortunately mostly associated with unforeseeable side-effects and therefore represent an incalculable risk.

In addition to treatment with medicaments, which tends to be undesirable, in particular in children, owing to the unforeseeable side-effects, surgical methods are also used for the therapy of urinary incontinence and in particular also of vesicoureteral reflux disease, for example the implantation of artificial sphincter (Lima et al., 1996, Combined use of enterocystopasty and a new type of artificial sphincter in the treatment of urinary incontinence, J. Urology, 156: 622-4), injection of collagens (Berman et al., 1997, Comparative cost analysis of collagen injection and facia lata sling cystourethropexy for the treatment of type III incontinence in women, J. Urology, 157: 122-4) and polytetrafluoroethylenes (Perez et al., 1996, Submucosal bladder neck injection of bovine dermal collagen for stress urinary incontinence in the pediatric population, Urology, 156: 633-6). As in the treatment of gastrooesophageal reflux disease (“GORD”), however, the use of injectable collagens has the disadvantageous effect here too that the treatment must frequently be repeated owing to the migration of the material (Khullar et al., 1996, GAX Collagen in the treatment of urinary incontinence in elderly women: A two year follow up. British J. Obtetrics & Gynecology, 104: 96-9) and can lead to the occurrence of allergies (McClelland and Delustro, 1996, Evaluation of antibody class in response to bovine collagen treatments in patients with urinary incontinence, J. Urology, 155: 2068-73).

In the therapy of the form of urinary incontinence that occurs in particular in children, vesicoureteral reflux disease, a surgical correction of the reflux is frequently undertaken, with all the known risks of a surgical operation. Although the condition of vesicoureteral reflux in children can improve or pass by itself in time, as indicated above, it leads in some cases to severe urinary tract and kidney infections and even to kidney failure. There is therefore a need in such cases in particular for a reliable, effective, minimally invasive and long-term method of treating this reflux disease. The endoscopic treatment method has various advantages over conventional surgical methods: it is an out-patient treatment, it does not lead to scarring, it involves a low risk of post-operative obstruction and it requires only a low outlay in terms of cost. Hitherto, various substances have already been proposed for submuscular injection (Teflon®, Polydimethylsiloxane®, Macroplastique®, collagen (bovine), Zyplast®, autologous chondrocytes, fatty tissue and blood). In addition, the preparation Deflux® (dextranomer/hyaluronic acid copolymer) has also been authorised in the meantime by the FDA (Oswald et al., 2002, Prospective comparison and 1-year follow-up of a single endoscopic subureteral polymethylsiloxane versus dextranomer/hyaluronic acid copolymer injection for treatment of vesicoureteral reflux in children, Urology 2002; 60: 894-7). However, it is found for all the proposed applications that the materials used hitherto for the therapy of urinary incontinence and in particular of vesicoureteral reflux disease have the disadvantage that inflammatory reactions are caused, the materials in some cases migrated, or multiple injections were necessary.

In summary, therefore, there is still a need for improved endogenous or exogenous filler materials which are able to overcome the above-mentioned disadvantages or particular requirements and are suitable in particular for the correction or treatment of wrinkles or volume defects as well as for the treatment of selected diseases, such as, for example, gastrooesophageal reflux disease, urinary incontinence and vesicoureteral reflux disease.

A solution to the problem outlined here should satisfy the following requirements in particular:

1. The material should readily be injectable via very thin cannulas (>27 gauge).

2. The material should form the desired volume at the site of implantation and maintain it in the long term.

3. The implant should acquire and retain the formed volume in its geometric form.

4. The material should remain at the injection site and not migrate.

5. The material must be biocompatible at the time of administration and during its life in vivo.

6. The material should preferably not contain either animal or synthetic or non-degradable constituents.

### SUMMARY OF THE INVENTION

The present invention relates to the use of an alginate in the preparation of an uncrosslinked, highly pure and high molecular weight alginate solution (sol) as a filler material in medicine, in particular (dermatological) surgery, or in cosmetics for the purpose of increasing volume, wherein the alginate is present in the uncrosslinked, highly pure and high molecular weight alginate solution (sol) in a concentration of from 0.5 to 2.5% (w/v) alginate solids content and the uncrosslinked, highly pure and high molecular weight alginate solution is injected in vivo and leads to spontaneous in situ Ca2+ crosslinking without the exogenous addition of a crosslinker. The present invention further describes the use of this uncrosslinked, highly pure and high molecular weight alginate solution (sol) for the treatment of wrinkles, for example on the hand, the face or the décolleté, or of volume deficiencies, for increasing the volume, for example in the case of (HIV-induced) lipoatrophy, of the breasts, and for the treatment of selected diseases, such as, for example, gastrooesophageal reflux disease, urinary incontinence or vesicoureteral reflux disease, by injection into the corresponding sphincter musculature, or for use in reconstructive surgery, in particular for cosmetic purposes.

### BRIEF DESCRIPTION OF THE DRAWINGS

The following figures are intended merely to explain the invention by way of example, without limiting it in any way.

FIG. 1 shows the explantation after administration of test substance 1 (uncrosslinked alginate solution) 60 minutes after administration; (A) shows the subcutaneously injected alginate solution which, even after removal of the skin, is visible macroscopically as a swelling; (B) shows that the applied test substance is readily palpable and dimensionally stable; and (C) shows that the once liquid alginate solution has crosslinked by means of endogenous calcium only 60 minutes after administration and can be explanted as a gel cushion.

FIG. 2 shows H&E stains of the sites of injection of the uncrosslinked, highly pure alginate solution (sol) prepared according to the invention, 6 months after subcutaneous injection in the rat; (A) shows an overview of an alginate deposit; and (B) shows a detail of an alginate deposit within the fatty tissue with embedded collagen fibres.

FIG. 3 shows collagen stains (van Gieson staining) of the injection sites with the test implants 1 (AL-018; A, B) and 2 (AL-019; C, D) four weeks after injection into the rabbit; (A) shows collagen fibres within the i.d. injected test implant 1; the collagen fibres are uniformly ruby-red in colour, like the original dermal collagen bundles; (B) shows a detailed photomicrograph of the s.c. injected test implant 1, which is interspersed with collagen fibres; (C) shows a s.c. injected test implant 2 which is interspersed with a large number of collagen fibres; and (D) describes a detailed photomicrograph of the s.c. injected test implant 2, which is interspersed with collagen fibres of various lengths.

FIG. 4 shows the ejection pressure of 1 ml of alginate solution by means of a 30G cannula. As will readily be seen, the pressure is uniform over the entire range (with the exception of the beginning and the end, owing to physical circumstances), that is to say the uncrosslinked, highly pure alginate solution (sol) prepared according to the invention can be ejected from a 30G cannula with a relatively low pressure, that is to say comparatively easily.

FIG. 5 shows the mean values of the palpated maximum and minimum diameters of the various test implants (produced by injection of the test substances or implantation of the reference substances) over the test period. The implant was palpated in the narcotised animal (rat) and the size of the implant was measured by means of a slide gauge. Test substance 1 (uncrosslinked high molecular weight alginate solution), Test substance 2 (low molecular weight uncrosslinked alginate solution), Reference substance 1 (CellBeads® 500), Reference substance 2 (CellBeads® 500, autoclaved). The data are derived from the measurement of the following implant sites: day 1 n=16, day 2-7 in each case n=12, day 14-28 in each case n=8, remaining days in each case n=4.

### DETAILED DESCRIPTION OF THE INVENTION

The problem is solved by the present invention and the accompanying claims. In particular, the present invention relates to the use of an alginate in the preparation of an uncrosslinked, highly pure and high molecular weight alginate solution (sol) as a filler material in medicine, in particular surgery, and (invasive) cosmetics for the purpose of increasing volume, wherein the alginate is present in the uncrosslinked, highly pure and high molecular weight alginate solution (sol) in a concentration in particular of approximately from 0.5 to 2.5% (w/v) alginate solids content, and the uncrosslinked, highly pure and high molecular weight alginate solution is injected in vivo and leads to spontaneous Ca2+ crosslinking without the exogenous addition of a crosslinker. Accordingly, the above-mentioned alginate solution is suitable for being crosslinked after injection, without the exogenous addition of a crosslinker.

Within the scope of the present invention, the filler material formed in situ by the use according to the invention is preferably a (relatively) solid, high molecular weight alginate implant with long-term stability, which is also referred to as a monolithic alginate implant. Also preferably, the monolithic alginate implant prepared by the use according to the invention is a gel body. Within the scope of the present invention, monolithic alginate implants with long-term stability are preferably also monolithic alginate implants that are still found at the injection site to a considerable extent (at least over 50% of the initial volume) even after (at least) 6 months.

Within the scope of the present invention, an alginate is a naturally occurring, anionic, unbranched polysaccharide which is conventionally isolated from marine brown algae and for the degradation of which the human body has no enzymes—unlike, for example, in the case of hyaluronic acid. It is composed of homopolymeric groups of beta-D-mannuronic acid and alpha-L-guluronic acid, separated by heteropolymeric regions of both acids. The commercial alginates which are today already obtained in large amounts are used industrially (e.g. in paper production) and as a food additive (E numbers 400-405) (e.g. Askar, 1982, Alginate: Herstellung, Eigenschaften and Verwendung in der Lebensmittel-industrie. Alimenta 21: 165-8). However, they are increasingly also being used in pharmacy, medicine and biotechnology. They are routinely used as a constituent of wound dressings (Gilchrist and Martin, 1983, Wound treatment with Sorbsan—an alginate fibre dressing. Biomaterials 4: 317-20; Agren, 1996, Four alginate dressings in the treatment of partial thickness wounds: A comparative experimental study. Br. J. Plast. Surg. 49: 129-34). Alginates have been and are also used in a large number of tissue engineering and drug delivery projects (e.g. Uludag et al., 2000, Technology of mammalian cell encapsulation. Advanced Drug Delivery Reviews 42: 29-64). The deciding property of alginates for use in biotechnology and in medicine is their capability for ionotropic gel formation. The alkali salts of alginates are water-soluble, while the salts of alginates with most di- or poly-valent cations form insoluble gels (so-called hydrogels) in aqueous solution. The large physical breadth of variation of the alginates is due to a number of factors: viscosity (or molar mass distribution), concentration, ratio of the monomers and the affinity of the cation typically used for the crosslinking.

The biocompatibility of the alginate used within the scope of the present invention depends substantially on its purity, especially on the absence of exogenous proteins and fragments thereof. Therefore not all alginates are suitable for the use described herein, because they can contain impurities which can cause an immune defence reaction following implantation in humans, for example fibrosis or inflammatory reactions (Zimmermann et al., 1992, Production of mitogen-contamination free alginates with variable ratios of mannuronic acid to guluronic acid by free flow electrophoresis, Electrophoresis 13: 269-74). Preference is therefore given to the use of those alginates which do not contain such impurities, particularly preferably highly pure and high molecular weight alginates, and yet more preferably highly pure, high molecular weight potassium or sodium alginate. Within the scope of the present invention there is therefore prepared an uncrosslinked, highly pure and high molecular weight alginate solution (sol) present in liquid form, which preferably does not cause any kind of immune reaction of the innate or adaptive immune system of a patient. Such an uncrosslinked, highly pure and high molecular weight alginate solution (sol), present in liquid form, that is to be prepared can be isolated by the use of homogeneous algae raw material and standard methods (Jork et al., 2000, Biocompatible alginate from freshly collected Laminaria pallida for implantation, Appl. Microbiol. Biotechnol. 53: 224-229) according to DE 198 36 960. The requirements for biocompatibility are thereby met.

Highly pure alginates are typically understood as being chemically highly pure alginates. Chemically highly pure alginates should typically not exceed upper limits in terms of their heavy metal or endotoxin content. Preferably, the upper limit of the heavy metal content is <50 ppm, most particularly preferably <30 ppm. The endotoxin content of chemically highly pure alginates (this means in particular the occurrence of lipopolysaccharides) should preferably be <200 IU/g dry material, most particularly <100 IU/g dry material, and yet more preferably <50 IU/g dry material. In a particular embodiment, chemically highly pure alginate also exhibits upper limits in terms of the amino acid content, particularly preferably in this connection with an amino acid content of <10 ng/mg dry material, yet more preferably <5 ng/mg dry material and still more preferably <3 ng/mg dry material. In a further preferred embodiment, chemically highly pure alginate additionally has a limited atomic sulfur content. The content of atomic sulfur in such a case is typically <200 ppm, most particularly preferably <100 ppm.

The alginate of the uncrosslinked, highly pure and high molecular weight alginate solution (sol) prepared according to the invention crosslinks in situ after administration to a patient, that is to say in vivo, by a Ca2+-induced crosslinking which takes place spontaneously, so that monolithic alginate implant bodies crosslinked in vivo, that is to say after administration, form at the injection site. Contrary to what has hitherto been described in the literature, for example in WO 2005/105167 (Cellmed AG, Germany), in WO 2006/044342 or in US 2006/0159823, it has been found according to the invention, wholly surprisingly, that the spontaneous crosslinking which takes place in the body without the exogenous addition of divalent ions, such as, for example, Ca2+ or Ba2+, can result in an implant with long-term stability if high molecular weight alginate is used. WO 2005/105167, on the other hand, teaches that the long-term stability in vivo is substantially dependent on the cationic crosslinking and that the gel crosslinked ex vivo by addition of divalent Ca2+ or Ba2+ ions slowly decrosslinks by slow cation exchange for the monovalent ions Na+ or K+ typically occurring in natural tissues and cells, and thus dissolves. In contrast, it has now been possible to show, by means of the present invention, that the in vivo stability of the monolithic alginate implant body formed according to the invention is dependent on the molecular weight and not on the crosslinking. Furthermore, it has been taught, for example in WO 2005/105167, that, when uncrosslinked alginate sol is injected, the uncrosslinked alginate sol is absorbed and disappears within a few days or weeks. This is in fact true for low molecular weight alginate sols, but not in the case of the choice according to the invention of a high molecular weight alginate. In order to obtain implants that are stable for as long as possible, alginates having as high a molecular weight as possible are therefore preferably used according to the present invention.

Within the scope of the present invention, therefore, the uncrosslinked, highly pure and high molecular weight alginate solution (sol), present in liquid form, typically contains highly pure and high molecular weight alginates having a mean molar mass of more than 200,000 Daltons, preferably more than 250,000 Daltons, more preferably more than 300,000 Daltons, yet more preferably more than 400,000 Daltons, for example preferably highly pure and high molecular weight alginates having a mean molar mass of from 200,000 to 500,000 Daltons, more preferably highly pure and high molecular weight alginates having a mean molar mass of from 200,000 (or 300,000 or 400,000) to 1,000,000 Daltons, and most preferably highly pure and high molecular weight alginates having a mean molar mass of from 200,000 (or 300,000 or 400,000) to 5,000,000 Daltons.

The average molecular weight of the alginates can be determined by standard methods, for example those described in Ueno et al., 1988, Chem. Pharm. Bull. 36, 4971-4975; Wyatt, 1993, Anal. Chim. Acta; 1-40; and Wyatt Technologies, 1999, “Light scattering University DAWN Course Material” and “DAWN EOS Manual” Wyatt Technology Corporation, Santa Barbara, Calif., USA.

The viscosity of a 0.2% (w/v) alginate solution (sol) (prepared according to the invention, present in liquid form, uncrosslinked, highly pure and high molecular weight) in 0.9% sodium chloride solution can conventionally be from 3 to 100 mPa s; it is preferably from 20 to 30 mPa s. Such a choice of viscosity permits in particular the choice of a very thin cannula for injection, as described hereinbelow.

The concentration of the alginate for the preparation of the uncrosslinked, highly pure and high molecular weight alginate solution (sol) prepared according to the invention is typically from 0.5 to 2.5% (w/v) alginate solids content and more preferably in a concentration from 0.6 to 1.0% (w/v) alginate solids content, based on the total weight of the uncrosslinked, highly pure and high molecular weight alginate solution (sol) present in liquid form. The content of alginates in the uncrosslinked, highly pure and high molecular weight alginate solution (sol) prepared according to the invention can be determined by methods known to a person skilled in the art.

The uncrosslinked, highly pure and high molecular weight alginate solution (sol) prepared according to the invention preferably further contains only such small amounts or traces of divalent ions, for example of a divalent ion from the group of the alkaline earth metals, such as, for example, Mg2+, Ca2+, Ba2+, etc., to prevent premature (that is to say in particular spontaneous) polymerisation of the alginate solution (sol), particularly preferably no amounts or traces of such divalent ions. For example, the uncrosslinked, highly pure and high molecular weight alginate solution (sol) prepared according to the invention can contain up to about 0.5 mg/l Ca2+ ions (corresponding to about 0.00005% Ca2+) and/or 0.05 mg/l Mg2+ ions (corresponding to about 0.000005% Mg2+) without premature polymerisation of the alginate solution (sol) occurring. In other words, the uncrosslinked, highly pure and high molecular weight alginate solution (sol) prepared according to the invention crosslinks in situ solely on account of the Ca2+ ions occurring physiologically in vivo or optionally on account of other divalent ions occurring physiologically in vivo, and therefore does not require the exogenous addition of a crosslinker. Other divalent crosslinkers also occur in the uncrosslinked, highly pure and high molecular weight alginate solution (sol) prepared according to the invention preferably only in such a low concentration that crosslinking of the alginate solution (sol) does not take place prematurely. However, it is particularly preferred for the uncrosslinked, highly pure and high molecular weight alginate solution (sol) prepared according to the invention not to contain such other divalent crosslinkers.

According to a further embodiment, the uncrosslinked, highly pure and high molecular weight alginate solution (sol) prepared according to the invention also comprises other constituents, such as, for example, a (physiological) injection solution, active ingredients and/or further fillers. The uncrosslinked, highly pure and high molecular weight alginate solution (sol) prepared according to the invention can also contain as a further constituent substances that additionally prevent migration of the monolithic alginate implant body formed in situ, for example that permit better anchoring of the monolithic alginate implant body at the injection/transplantation site. The addition of these constituents to the uncrosslinked, highly pure and high molecular weight alginate solution (sol) prepared according to the invention can take place, as required, by the treating doctor or previously during preparation of the uncrosslinked, highly pure and high molecular weight alginate solution (sol) prepared according to the invention.

The alginate of the uncrosslinked, highly pure and high molecular weight alginate solution (sol) prepared according to the invention can be dissolved in a (physiological) injection solution. Such a (physiological) injection solution typically includes any injection solution which can be used in the prior art, for example a (physiological) injectable sodium chloride solution, a (physiological injectable) potassium chloride solution, a solution containing sodium chloride, potassium chloride and optionally sodium acetate, or optionally a Ringer's or Ringer's lactate solution, etc., such solutions preferably also containing only such small amounts or traces of divalent ions (or other crosslinkers), for example divalent ions from the group of the alkaline earth metals, such as, for example, Mg2+, Ca2+, Ba2+, etc., to prevent premature polymerisation of the alginate solution (sol), particularly preferably no amounts or traces of such divalent ions (or other crosslinkers).

According to a further embodiment of the present invention, the uncrosslinked, highly pure and high molecular weight alginate solution (sol) prepared according to the invention contains substances that additionally prevent migration of the monolithic alginate implant body formed in situ. Although diffusion or migration of the filler material into the surrounding tissue is already advantageously prevented by the monolithic alginate implant body formed in situ itself. The reason for this property is, on the one hand, that the monolithic alginate implant body is a one-piece implant which is comparatively large and immovable compared with the surrounding tissue. On the other hand, the implant is additionally a soft, elastic form which, as such, is less readily displaced in the tissue, whereas hard forms can more readily be displaced in the tissue. In order to enhance this effect even further, it is therefore possible to add to the uncrosslinked, highly pure and high molecular weight alginate solution (sol) prepared according to the invention also substances (which bind in situ, for example covalently, to the alginate and) which, after injection, effect further histological binding of the monolithic alginate implant body formed in situ with the surrounding tissue and thus prevent migration. Such substances include, for example, adhesion proteins (e.g. RGD tripeptides).

According to a further embodiment, the uncrosslinked, highly pure and high molecular weight alginate solution (sol) prepared according to the invention additionally contains active ingredients which, without implying any limitation, are selected from pharmaceutically active compounds, nutrients, marker substances and vital cells, for example endogenous autologous cells (of the patient to be treated), for example from liposuction. In this connection, pharmaceutically active compounds can be selected from the substance classes of the vitamins, adhesion proteins, anti-inflammatory substances, antibiotics, analgesics, growth factors, hormones, particularly preferably selected from protein- and/or peptide-based active ingredients, such as, for example, human growth hormone, bovine growth hormone, porcine growth hormone, growth hormone releasing hormone/peptide, granulocyte-colony stimulating factor, granulocyte macrophage-colony stimulating factor, macrophage-colony stimulating factor, erythropoietin, bone morphogenic protein, interferon or derivatives thereof, insulin or derivatives thereof, atriopeptin-III, monoclonal antibodies, tumour necrosis factor, macrophage activating factor, interleukin, tumour degenerating factor, insulin-like growth factor, epidermal growth factor, tissue plasminogen activator, factor MV, factor IMV and urokinase.

The uncrosslinked, highly pure and high molecular weight alginate solution (sol) prepared according to the invention can further additionally contain a water-soluble auxiliary substance in order to stabilise the active ingredients, for example a protein, such as, for example, albumin or gelatin; an amino acid, such as, for example, glycine, alanine, proline, glutamic acid, arginine, or a salt thereof; carbohydrates, such as, for example, glucose, lactose, xylose, galactose, fructose, maltose, sucrose, dextran, mannitol, sorbitol, trehalose and chondroitin sulfate; an inorganic salt, such as, for example, phosphate; a wetting agent, such as, for example, TWEEN® (ICI), polyethylene glycol, or a mixture thereof.

The above-mentioned active ingredients are typically introduced in a manner known in the prior art during the preparation of the uncrosslinked, highly pure and high molecular weight alginate solution (sol) described herein. The active ingredients are preferably so chosen that handling of the uncrosslinked, highly pure and high molecular weight alginate solution (sol) prepared according to the invention is not impaired (for example the viscosity of the solution), that is to say it is possible in particular to use standard, for example, 27, 30 or 33 gauge, cannulas, as described herein.

According to another embodiment, the uncrosslinked, highly pure and high molecular weight alginate solution (sol) prepared according to the invention contains at least one further substance having filler properties in addition to the alginate. When the at least one further substance having filler properties occurs in the uncrosslinked, highly pure and high molecular weight alginate solution (sol), the at least one further substance having filler properties is suspended in the alginate solution (sol) preferably in an amount by weight of from 5 to 50% of the total filler weight (w/w), more preferably in an amount by weight of from 5 to 40% of the total filler weight (w/w), even more preferably in an amount by weight of from 5 to 30% of the total filler weight (w/w) and most preferably in an amount by weight of from 5 to 20% of the total filler weight (w/w).

The at least one further substance having filler properties in the uncrosslinked, highly pure alginate solution (sol) prepared according to the invention is preferably selected, without implying any limitation, from the group consisting of hyaluronic acid, collagen and polyacrylamide in soluble form. If the at least one further substance having filler properties is hyaluronic acid, the hyaluronic acid is preferably present in the uncrosslinked, highly pure and high molecular weight alginate solution (sol) prepared according to the invention in the form of soluble, uncrosslinked, highly purified hyaluronic acid in a concentration of from 5 to 50%, based on the total filler weight (w/w).

Within the context of the present invention, the hyaluronic acid can be selected from hyaluronic acid or a salt thereof, for example from sodium hyaluronate, potassium hyaluronate or ammonium hyaluronate, typically having a molecular weight in the range from 10,000 Da to 10,000,000 Da, preferably having a molecular weight in the range from 25,000 Da to 5,000,000 Da and most preferably having a molecular weight in the range from 50,000 Da to 3,000,000 Da.

According to a particular embodiment, the hyaluronic acid or a salt thereof can have a molecular weight in the range from 300,000 Da to 3,000,000 Da, preferably a molecular weight in the range from 400,000 Da to 2,500,000 Da, more preferably a molecular weight in the range from 500,000 Da to 2,000,000 Da and most preferably a molecular weight in the range from 600,000 Da to 1,800,000 Da.

According to another particular embodiment, the hyaluronic acid or a salt thereof can have a molecular weight in the range from 10,000 Da to 800,000 Da, preferably a molecular weight in the range from 20,000 Da to 600,000 Da, more preferably a molecular weight in the range from 30,000 Da to 500,000 Da, yet more preferably a molecular weight in the range from 40,000 Da to 400,000 Da and most preferably a molecular weight in the range from 50,000 Da to 300,000 Da.

The amount of hyaluronic acid or of a salt thereof can be determined by methods known to a person skilled in the art, for example by means of the carbazole process (Bitter and Muir, 1962, Anal. Biochem. 4: 330-334). The average molecular weight of the hyaluronic acid or a salt thereof can likewise be determined by standard methods, for example those described in Ueno et al., 1988, Chem. Pharm. Bull. 36, 4971-4975; Wyatt, 1993, Anal. Chim. Acta; 1-40; and Wyatt Technologies, 1999, “Light scattering University DAWN Course Material” and “DAWN EOS Manual” Wyatt Technology Corporation, Santa Barbara, Calif., USA.

Equally preferably, the at least one further substance having filler properties in the uncrosslinked, highly pure and high molecular weight alginate solution (sol) prepared according to the invention can be an insoluble substance (having filler properties).

The insoluble substance (having filler properties) preferably has a particle size of from 10 to 150 μm. Particularly preferably, the particle form of these insoluble substances is substantially round, with a diameter of from 10 to 80 μm. In the present case, “substantially round” means in particular a form that is similar in the broadest sense to a spherical form. In this connection, the insoluble substances in the uncrosslinked, highly pure and high molecular weight alginate solution (sol) prepared according to the invention are preferably selected from the group consisting of calcium hydroxylapatite, polymethyl methacrylate (PMMA), for example in the form of PMMA microparticles, poly-L-lactic acid microparticles, HEMA particles, calcium hydroxylapatite particles, etc.

Also preferably, the above-described insoluble substances (having filler properties) are fibrous. Such staple fibres preferably have a diameter of from 1 to 80 μm and a length of from 10 to 200 μm. Yet more preferably, the staple fibres have a diameter of from 5 to 40 μm and a length of from 20 to 100 μm. The fibres typically consist of tissue-compatible polymers which can be broken down in the body and are preferably selected, without implying any limitation, from the group consisting of fibres of collagen, polylactic acid and copolymers thereof (with glycine), covalently crosslinked hyaluronic acid, alginic acid, acrylic and methacrylic acid ester polymers and copolymers thereof.

Alternatively or in addition, the at least one further substance having filler properties in the uncrosslinked, highly pure and high molecular weight alginate solution (sol) prepared according to the invention can be selected from the group consisting of autologous constituents, including cells, for example endogenous autologous cells (of the patient to be treated), plasma proteins or liposuction material, etc.

The uncrosslinked, highly pure and high molecular weight alginate solution (sol) prepared according to the invention can contain one or more of the above-mentioned constituents, provided that the constituents are mutually biocompatible, chemically stable and do not exhibit any (disruptive) interactions with one another or in respect of the alginate that is present or the monolithic alginate implant that is formed. In particular, it is preferred that the uncrosslinked, highly pure and high molecular weight alginate solution (sol) prepared according to the invention does not crosslink wholly or partially prior to administration to a patient owing to one or more of the above-mentioned constituents. Nor must the further constituents typically have the effect that the viscosity of the uncrosslinked, highly pure and high molecular weight alginate solution (sol) prepared according to the invention is outside the range described above as being preferred.

The uncrosslinked, highly pure and high molecular weight alginate solution (sol) prepared according to the invention is usually prepared and introduced into containers under sterile conditions. Alternatively or in addition, the uncrosslinked, highly pure and high molecular weight alginate solution (sol) prepared according to the invention can be sterilised at the end by means of a suitable method according to the prior art, as long as no reduction of the volume takes place to a considerable extent. The uncrosslinked, highly pure and high molecular weight alginate solution (sol) prepared according to the invention can also be stored in the frozen state. The rapid, uncomplicated and also sterile preparation of the uncrosslinked, highly pure and high molecular weight alginate solution (sol) prepared according to the invention is a fundamental advantage of the present invention over the materials shown in the prior art, in particular the preparation of beads/particles.

According to a particular embodiment of the present invention, the invention includes the use of an alginate in the preparation of an uncrosslinked, highly pure and high molecular weight alginate solution (sol) as described herein for the treatment of wrinkles, especially in the region of the face, for example in the region of the facial muscles, and of the décolleté, the hands; for the treatment of volume deficits, in particular for increasing the volume, for example in the case of lipoatrophy, of the breasts, for example after mammary carcinoma or breast augmentation surgery, etc.; and in particular for cosmetic purposes, or for the treatment of selected diseases, such as, for example, gastrooesophageal reflux disease, urinary incontinence or vesicoureteral reflux disease, for example by supporting sphincter musculatures by injection into the corresponding sphincter musculature, for use in reconstructive surgery, or in tumour therapy.

The present invention describes inter alia also the use of an alginate in the preparation of an uncrosslinked, highly pure and high molecular weight alginate solution (sol) as a filler material in medicine, in particular (dermatological) surgery and cosmetics (for example for the purpose of increasing volume), wherein the alginate is present in the uncrosslinked, highly pure and high molecular weight alginate solution (sol) in a concentration of approximately from 0.5 to 2.5% (w/v) alginate solids content and the uncrosslinked, highly pure and high molecular weight alginate solution is injected in vivo and leads to spontaneous in situ Ca2+ crosslinking without the exogenous addition of a crosslinker. In particular, the present invention describes the use of this uncrosslinked, highly pure and high molecular weight alginate solution (sol) for the treatment of wrinkles, especially in the region of the face, for example in the region of the facial muscles, and of the décolleté, the hands; for the treatment of volume deficits, in particular for increasing the volume, for example in the case of lipoatrophy, of the breasts, for example after mammary carcinoma or breast augmentation surgery, etc.; and in particular for cosmetic purposes, or for the treatment of selected diseases, such as, for example, gastrooesophageal reflux disease, urinary incontinence or vesicoureteral reflux disease, for example by supporting sphincter musculatures by injection into the corresponding sphincter musculature, for use in reconstructive surgery, in tumour therapy.

The uncrosslinked, highly pure and high molecular weight alginate solution (sol) prepared according to the invention is typically administered to the patient in liquid form by means of injection. Administration of the uncrosslinked, highly pure and high molecular weight alginate solution (sol) prepared according to the invention preferably takes place by transdermal, intradermal, subdermal, subcutaneous or intramuscular injection into a suitable injection site of the patient. The choice of the injection site and the injection volume to be administered depend on the condition to be treated or on the disease to be treated, particularly preferably the conditions or diseases to be treated as described herein.

Administration of the uncrosslinked, highly pure and high molecular weight alginate solution (sol) prepared according to the invention usually takes place by injection using cannulas having a diameter of typically from 20 to 33 gauge, preferably from 26 to 33 gauge. The uncrosslinked, highly pure and high molecular weight alginate solution (sol) prepared according to the invention should therefore preferably be administrable by means of a syringe having a cannula of the above-mentioned type. Commercial cannulas, for example having a diameter of from 27 to 33 gauge, are particularly preferred. Alternatively, the uncrosslinked, highly pure and high molecular weight alginate solution (sol) prepared according to the invention can be administered by means of suitable other techniques which are known in the prior art, for example by the use of endoscopic or laparoscopic techniques. The injection can take place by a single injection, repeated injection or multiple injections into the same or different (typically adjacent) areas, for example of the skin or of the sphincter musculature.

The injection volume of the uncrosslinked, highly pure and high molecular weight alginate solution (sol) prepared according to the invention that is to be administered is usually in a range from 0.1 to 100 ml, preferably in a range from 0.1 to 50 ml, more preferably in a range from 0.1 to 40 ml, yet more preferably in a range from 0.1 to 30, 20 or 10 ml, and most preferably in a range from 0.1 to 1, 2 or 5 ml. The amount of the injection volume to be administered can likewise be over 100 ml if, for example, large volume deficits are to be filled. The requirement for the choice of the injection volume to be administered is typically that the tissue to be treated is so perfused with blood and/or supplied with Ca2+ ions that crosslinking of the uncrosslinked, highly pure and high molecular weight alginate solution (sol) prepared according to the invention can take place in situ, that is to say in the tissue, within a limited period of time, for example a period of not more than several hours (from 0 to 24 hours, preferably from 0 to 10 hours, more preferably from 0 to 1, 2 or 5 hours). By suitable means, the treating doctor can ensure that the patient's Ca2+ metabolism is balanced prior to therapy, in parallel therewith or in a subsequent treatment. Such methods preferably do not include the co-administration of crosslinkers and alginate solution (sol) described in the prior art but optionally use other methods, such as, for example, an appropriate diet, the oral intake of calcium-rich products, etc. The choice of the injection volume to be administered is also dependent on the judgement of the treating doctor. A safe and effective amount of the uncrosslinked, highly pure and high molecular weight alginate solution (sol) prepared according to the invention is thus typically administered. As used herein, “safe and effective amount” means an amount of the uncrosslinked, highly pure and high molecular weight alginate solution (sol) prepared according to the invention that is sufficient to bring about a significant change in the condition or disease to be treated but small enough to avoid serious side-effects (with a reasonable advantage/risk ratio), that is to say within the range of reasonable medical judgement. A safe and effective amount of the uncrosslinked, highly pure and high molecular weight alginate solution (sol) prepared according to the invention will therefore typically vary in association with the particular condition or disease to be treated and with the age and physical condition of the patient to be treated, the severity of the condition, the duration of treatment, the nature of any accompanying therapy and similar factors, within the knowledge and experience of the accompanying doctor. The uncrosslinked, highly pure and high molecular weight alginate solution (sol) prepared according to the invention can be used for both human and veterinary medical purposes.

According to a first alternative, the uncrosslinked, highly pure and high molecular weight alginate solution (sol) prepared according to the invention is used for the treatment of wrinkles. The treatment of wrinkles conventionally includes the treatment of skin deficits caused, for example, by ageing, environmental influences, weight loss, pregnancy, diseases, in particular HIV infection, surgical operations and acne. In particular, the treatment of wrinkles includes the treatment of facial wrinkles, in particular in the region of the facial muscles, the treatment of forehead wrinkles, frown wrinkles, worry wrinkles, drooping eyelids, crow's-feet, nasolabial folds, the use for injecting the lips, and the treatment of wrinkles in the region of the hands and the décolleté. Treatment is typically carried out by intra- or sub-dermal injection of the affected skin area. To that end, the uncrosslinked, highly pure alginate solution (sol) prepared according to the invention is preferably injected through a syringe having a cannula diameter of from 20 to 33 gauge, more preferably from 26 to 33 gauge. Alternatively, the uncrosslinked, highly pure alginate solution (sol) prepared according to the invention can be administered by other suitable techniques known in the prior art. Injection can be carried out by means of a single injection, repeated injection or multiple injections into the same or different (e.g. adjacent) areas of the skin. In the treatment of wrinkles, preferably only a small volume, more preferably in a range from 0.1 ml to 10 ml, yet more preferably in a range from 0.1 ml to 5, 2 or only 1 ml, is transferred with each puncture, until the desired total volume has been injected. Laminar support (plumping) and tightening of the skin is thus achieved, which results in the disappearance or partial disappearance of the wrinkles and/or volume deficits in the corresponding area. The injection can also be carried out once, repeatedly or many times, so that a volume of from 0.1 ml to 10 ml or even more is ultimately applied. Application is thereby effected, usually at each individual administration, preferably by slowly withdrawing the injection cannula/needle while at the same time injecting the volume. This method of injection is particularly suitable in the case of deeper wrinkles. The use according to the invention for wrinkle injection as described herein can at any time be combined with conventional treatment methods. Such treatment methods include, for example, conventional surgical and/or medical treatment methods.

According to a second alternative, the uncrosslinked, highly pure and high molecular weight alginate solution (sol) prepared according to the invention is used for the treatment of gastrooesophageal reflux disease. Typically, treatment within the scope of the present invention is carried out by injection of the uncrosslinked, highly pure and high molecular weight alginate solution (sol) prepared according to the invention into the wall regions of the lower oesophageal sphincter muscle or the surrounding tissue. The sphincter volume thereby increases proportionally to the volume of the uncrosslinked, highly pure and high molecular weight alginate solution (sol) prepared according to the invention that is injected. The inner lumen of the sphincter muscle is thereby reduced and thus permits better contraction of the muscle and accordingly prevents the stomach acid from escaping into the oesophagus. The injection is preferably to be carried out by standard techniques corresponding to the prior art, such as, for example, direct injections or by the use of endoscopic or laparoscopic techniques. The use according to the invention described here for the treatment of gastrooesophageal reflux disease can also be combined with conventional treatment methods. Such treatment methods include, for example, conventional surgical and/or medical treatment methods.

According to a third alternative, the uncrosslinked, highly pure and high molecular weight alginate solution (sol) prepared according to the invention can be used to treat urinary incontinence and vesicoureteral reflux disease. The use of the uncrosslinked, highly pure alginate solution (sol) prepared according to the invention is also suitable in the case of the temporary, non-chronic occurrence of forms of urinary incontinence and vesicoureteral reflux disease. The treatment of these diseases is typically carried out by injection of the uncrosslinked, highly pure alginate solution (sol) prepared according to the invention into the urethral sphincter, the bladder sphincter or the urinary tract musculature. The sphincter volume thereby increases proportionally to the volume of the uncrosslinked, highly pure alginate solution (sol) prepared according to the invention that is injected. As a result, the inner lumen of the sphincter muscle is reduced here too and accordingly permits better contraction of the muscle, as a result of which the likelihood of urinary incontinence falls. Here too, the injection is preferably to be carried out by standard techniques corresponding to the prior art, such as, for example, direct injections or by the use of endoscopic or laparoscopic techniques. The use according to the invention described herein for the treatment of urinary incontinence and vesicoureteral reflux disease can likewise be combined with conventional treatment methods. Such treatment methods include, for example, conventional surgical and/or medical treatment methods.

According to a fourth alternative, the uncrosslinked, highly pure and high molecular weight alginate solution (sol) prepared according to the invention can be used in reconstructive surgery and in particular for cosmetic purposes. Such cases include, for example, cases in which a volume defect or volume deficit occurs in the tissue, for example if tumour tissue has been surgically removed or tissue is missing, leaving a cavity, that is to say a hollow space, in the tissue, which needs to be filled and/or is to be concealed for cosmetic reasons. Also included are cases in which structural (cosmetic) reconstruction of the affected body part or tissue is necessary following an accident, an operation or a disease such as, for example, HIV, etc.

According to a fifth alternative, the uncrosslinked, highly pure and high molecular weight alginate solution (sol) prepared according to the invention can therefore also be used for the treatment of facial (HIV-induced) lipodystrophy, in particular for the treatment of (HIV-induced) lipoatrophy. In connection with the present invention, (HIV-induced) lipoatrophy is a disease which is characterised especially by fatty tissue atrophy with regression of fatty tissue, for example as a result of treatment of HIV-infected patients with nucleoside reverse transcriptase inhibitors (NRTIs). Pathologically, this disease manifests itself in particular in the case of HIV infection in the region of the extremities, that is to say arms and/or legs, but also in the region of the buttocks and cheeks and, because of the rapid and in some cases frightening external change, it often leads to mental disturbances and consequently to social alienation in patients. Treatment of (HIV-induced) lipoatrophy can be effected by injecting the corresponding areas of the skin, especially in the facial region, for example in the region of the Zygomaticus major, etc. The choice of injection site and the amount of the uncrosslinked, highly pure and high molecular weight alginate solution (sol) prepared according to the invention that is to be administered are dependent on the severity of the disease and are typically within the judgement of the treating doctor, preferably within the values mentioned above.

According to a further, last alternative, the uncrosslinked, highly pure and high molecular weight alginate solution (sol) prepared according to the invention can also be used for the treatment of tumour diseases, for example by embolisation of the tumour. It is thereby possible in particular to block the vessels formed specifically by the tumour, which are to ensure the supply to the tumour, for example newly formed arteries, by injection of the uncrosslinked, highly pure and high molecular weight alginate solution (sol) prepared according to the invention into those vessels, as a result of which the supply to the tumour cells is cut off and the tumour cells die. In this connection, tumour diseases are selected, for example, without implying any limitation, from the group consisting of melanomas, malignant melanomas, colon carcinomas, lymphomas, sarcomas, blastomas, renal carcinomas, gastrointestinal tumours, gliomas, prostate tumours, bladder cancer, rectal tumours, stomach cancer, oesophageal cancer, pancreatic cancer, liver cancer, mammary carcinomas (=breast cancer), uterine cancer, cervical cancer, hepatomas, various virus-induced tumours, such as, for example, papilloma virus-induced carcinomas (e.g. cervical carcinoma=cervical cancer), adenocarcinomas, herpes virus-induced tumours (e.g. Burkitt's lymphoma, EBV-induced B-cell lymphoma), hepatitis B-induced tumours (hepatocellular carcinomas), HTLV-1- and HTLV-2-induced lymphomas, acusticus neurinoma, pulmonary carcinomas (=lung cancer=bronchial carcinoma), small cell lung carcinomas, pharyngeal cancer, anal carcinoma, glioblastoma, rectal carcinoma, astrocytoma, brain tumours, retinoblastoma, basalioma, medulloblastomas, vaginal cancer, testicular cancer, thyroid cancer, Hodgkin's syndrome, meningeomas, Schneeberg lung disease, hypophyseal tumour, carcinoids, neurinoma, spinalioma, Burkitt's lymphoma, laryngeal cancer, kidney cancer, thymoma, corpus carcinoma, bone cancer, non-Hodgkin's lymphomas, urethral cancer, tumours of the head and neck, oligodendroglioma, vulval cancer, intestinal cancer, colon carcinoma, oesophageal carcinoma (=oesophageal cancer), wart involvement, small intestine tumours, craniopharyngeomas, ovarian carcinoma, soft-part tumours, ovarian cancer (=ovarian carcinoma), pancreatic carcinoma (=pancreatic cancer), endometrial carcinoma, liver metastases, penile cancer, tongue cancer, gall bladder cancer, leukaemia, plasmocytoma, lid tumour, prostate cancer (=prostate tumours), etc.

Advantageously, in all the above-mentioned applications and alternatives, the monolithic alginate implant produced in situ by injection of the uncrosslinked, highly pure and high molecular weight alginate solution (sol) prepared and used according to the invention is biologically compatible and provides a good adhesive base for growth with cells or collagen fibres. If there should nevertheless be an incompatibility in an individual case, the monolithic alginate implant formed can optionally be dissolved again by injection of an EDTA or citrate solution or of a solution of other complex formers, preferably directly into the monolithic alginate implant that has formed.

Finally, the present invention also provides kits comprising the uncrosslinked, highly pure alginate solution (sol) prepared according to the invention, optionally one or more of the injection cannulas for administration described herein, and optionally instructions for use.

### ADVANTAGES OF THE INVENTION

The use of an alginate for the preparation of an uncrosslinked, highly pure and high molecular weight alginate solution (sol) as described herein as a filler material in medicine, in particular surgery, and cosmetics for the purpose of increasing volume has considerable advantages over the prior art. The primary advantages of the present invention can be summarised as follows:

A) Simple injection of the liquid, uncrosslinked, highly pure and high molecular weight alginate solution (sol) prepared according to the invention at a low injection pressure.
(I.) Fundamental advantages over the co-injection of alginate sol and Ca2+ or Ba2+ salt are in particular:
   1) Standard injection cannulas/needles of up to 33 gauge can be used.
   2) Direct use of a simple pre-filled syringe without a double chamber, mixing chamber or double cannula.
   3) As a result, simpler, more precise handling as regards injection site and volume.
   4) No impairment of injectability as a result of premature crosslinking in the needle.
   5) It is readily possible to add other polymers, fillers, active ingredients and, especially, cells, in particular autologous cells, in situ.
(II.) Fundamental advantages over the injection of bead preparations are in particular:
   1) Aseptic preparation of the alginate solution (sol) is much simpler and, especially, cheaper as compared with the preparation of beads or particles.
   2) Better shaping of the implant body because it is present monolithically in the form of the injection volume.
   3) Omission of the production steps bead formation, crosslinking in a crosslinker bath, subsequent washing step.
   4) Simple admixture of third substances (see above).

(B) In situ formation of a stable, crosslinked, monolithic alginate implant body having precisely the desired geometry, which can be shaped during and shortly after injection.

(C) Formation of the stable implant volume corresponding to the injection volume without loss of volume and the necessity for subsequent correction.

(D) Formation of implants which have long-term stability and last (markedly) more than 6 months with the appropriate choice of alginate.

(E) Addition of other soluble active ingredients or polymers, such as hyaluronic acid, collagen and the like, is readily possible.

(F) Addition of solid, insoluble particles or microfibres of different in vivo stability, such as hydroxylapatite, polymethyl methacrylate (PMMA), poly-L-lactic acid microparticles, is also readily possible.

(G) The addition of autologous cells, proteins, fat, etc. is also readily possible, even in situ by the treating doctor.

In addition, and of greatest importance, is the far superior clinical result. Unlike bead implants, slow replacement of the implant volume by the ingrowth of collagen fibres can take place with the alginate sol injections according to the invention (see experimental section). Accordingly, the monolithic alginate implant is not only highly compatible with the surrounding tissue but is also an excellent placeholder for cells owing to its high permeability and compatibility. The long durability of the implant body ensures that the corresponding placeholder is present even for slowly growing tissue.

### EXAMPLES

The following examples are intended to explain the invention solely by way of example, without limiting it in any way.

1. Preparation of the 1% (w/v) Alginate Solution

0.25 g of dried, highly pure alginate (purity >99%, MW>500,000 g/mol) was introduced under clean room conditions into 25 ml of 0.9% NaCl solution. The closed test tube was then rotated at room temperature until the Na alginate had dissolved completely. After sterile filtration again (0.2 μm syringe filter), 1 ml syringes were each filled with 500 μl of the solution and the finished product (syringes) was stored at 5° C. plus/minus 3° C. A 30G needle was used for the injection.

2. Preparation of Alginate Capsules (Comparative Test)

The alginate solution was prepared as described in Example 1, only 0.15 g of the same alginate being introduced into 25 ml of NaCl solution. For the purposes of complete dissolution, rotation was again carried out in a closed vessel on a test tube rotating device until dissolution was complete.

After sterile filtration (0.2 μm filter), the solution was added dropwise, by means of a conventional dropping apparatus, with control of the drop size, into a precipitation bath consisting of a 100 mM CaCl2 solution or 20 mM BaCl2 solution.

The capsules (beads) were then washed 5 times with 10 ml of Ringer's solution. The resulting capsules (beads) having a mean diameter of 450 μm were subsequently taken up in Ringer's solution, and 1 ml prefilled syringes were each filled with 500 μl.

The prefilled syringes, which were prepared under sterile conditions, were stored at 5° C. plus/minus 3° C. A 21G needle was used for the injection.

3. Subcutaneous Testing on the Rat
a) Subcutaneous Implantation
   Rats recommended according to international guidelines for the testing of subcutaneous compatibility were chosen accordingly, prepared and injected subcutaneously, under anaesthesia, with the test substances and reference substances (liquid alginate solution and prepared beads, see preparation under Example 2).

To that end, each animal was injected subcutaneously at the 4 test sites with 0.40 plus/minus 0.05 ml of the various test substances and reference substances (liquid alginate solution and prepared beads, see preparation under Example 2) in such a manner that the product is distributed over a distance of 2 cm by withdrawal of the needle. During and after withdrawal, a corresponding elongated swelling was visible. The injection sites were then marked with a tattoo.

The condition of the animals and of the injection sites was examined every two days initially and later twice a week.
b) Explantation Immediately after Implantation

Administration of the test substances and reference substances resulted in a readily palpable swelling.

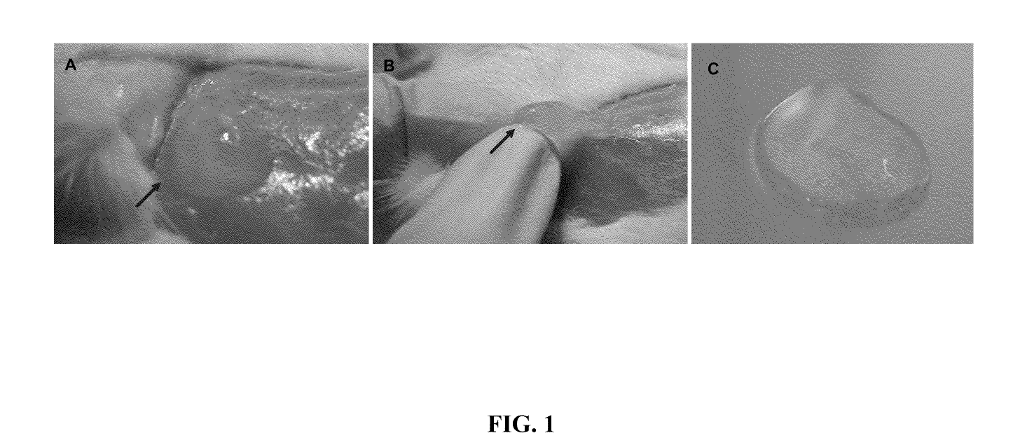

In an experiment conducted in addition to this study, the after injection of the highly pure, uncrosslinked and high molecular weight alginate solution prepared according to the invention (test substance 1) was explanted again 60 minutes after injection. At that time, the test substance had already been crosslinked by endogenous calcium and could be explanted as a gel cushion (see FIG. 1).

As will be seen in FIG. 1, administration of test substance 1 (uncrosslinked alginate solution) leads to a fully crosslinked implant 60 minutes after administration. The subcutaneously injected alginate solution is also visible macroscopically as a swelling after removal of the skin (see FIG. 1A). Furthermore, the administered test substance 1 is readily palpable and dimensionally stable (see FIG. 1B). The test additionally demonstrates that the once liquid alginate solution has been crosslinked by endogenous calcium only 60 minutes after administration and can be explanted as a gel cushion (see FIG. 1C).

4. Explantation and Pathology of the Implantation Sites

In each case 6 rats were euthanised after 7 days, 3 and 6 months. All the injection sites were opened and checked for the presence and nature of the implant.

The implants were examined visually and for Ca2+ content. After all three periods of time, all the implants were fully visible and well defined and could be isolated. Measurement of the Ca2+ content of the explanted beads was only possible as an approximation because the serum content of the explanted beads can be different.

The Ca2+ values of the monolithic implants formed from alginate solution (total volume approximately 0.4 ml) are equal to those of the externally Ca2+-crosslinked beads even at the 1st explantation after 7 days, and their Ca2+ content remains constant over the 6-month observation period (250-330 mg Ca2+/g implant).

The histopathological examination further showed that the monolithic implant is at least as tissue-compatible as the Ca2+-crosslinked beads.

Result after 1 Week:

After one week, the subcutaneous injection of the alginate solution into the rat exhibited a slightly inflammatory reaction (slight infiltration with macrophages, lymphocytes and plasma cells and, to a lesser degree, giant cells) in comparison to the implantation of beads, but lower infiltration with lymphocytes was observed as compared with the bead implantation. Polymorphonuclear cells and necrosis were not observed. Only slight fibrosis and fibroplasia was noted after injection of the alginate solution into the rat. Spiral-like fibres, evidently collagen fibres, can be detected within the alginate deposit.

Slight neovascularisation, scarcely detectable degradation and a fibrin inclusion could be observed after the injection of the alginate solution. There was no encapsulation of the alginate and no degeneration of the tissue as a result of the injection of the alginate solution.

Result after 3 Months:

The inflammatory reaction caused by the injected alginate solution was similar in nature after three months as after one week, with the exception that no giant cells occurred three months after injection.

The extent of fibroplasia and of fibrosis around the alginate deposit was reduced, and it was observed that, to a small degree, fibrotic tissue present forms a thin capsule with fibrin in two out of three cases. Similarly to the earlier time, slight neovascularisation was noted.

Result after 6 Months:

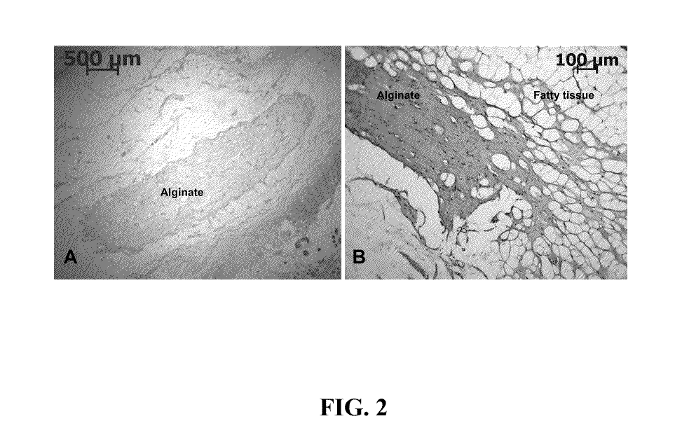

Six months after injection, the alginate was still present as a solid to network-like deposit with embedded collagen fibres (see FIG. 2) and exhibited good biocompatibility. Both the inflammation and the fibrotic reaction caused by the alginate implant were substantially better six months after injection of the alginate solution as compared with the biocompatibility after three months. None of the injection sites exhibited a fibrotic reaction, and the inflammatory response consisted only of a few plasma cells and macrophages. No further inflammatory responses were induced (see FIG. 2). FIG. 2 shows H&E stains of the sites of the injection of alginates 6 months after subcutaneous injection in the rat. FIG. 2A gives an overview of an alginate deposit, FIG. 2B shows a detail of a net-like outgrowth of the alginate deposit within the fatty tissue with embedded collagen fibres. Overall, therefore, the biocompatibility of the alginate implant crosslinked in situ solely by endogenous Ca2+ is evidently very good.

5. Subcutaneous Testing on the Rabbit

200 μl of a 1% highly pure alginate solution, prepared as described above according to the invention, was injected by means of a 1 ml syringe, using a 30G needle, into 6 injection sites on the left and right of the back of the shaved rabbit.

The aim of the testing was to assess the stability and local tolerability of two alginate preparations and a commercial comparison product of a chemically crosslinked particulate polysaccharine. The test design used here is recommended according to international guidelines for tests of this type.

6. Results of the Tests from Example 5

The injection sites removed following euthanasia after 4 and 12 weeks were examined histopathologically. At all the injection sites, alginate hydrogel bodies were embedded in the subcutaneous tissue of the test animals. The material exhibited a high degree of tissue integration and very good tolerability of the test implants.

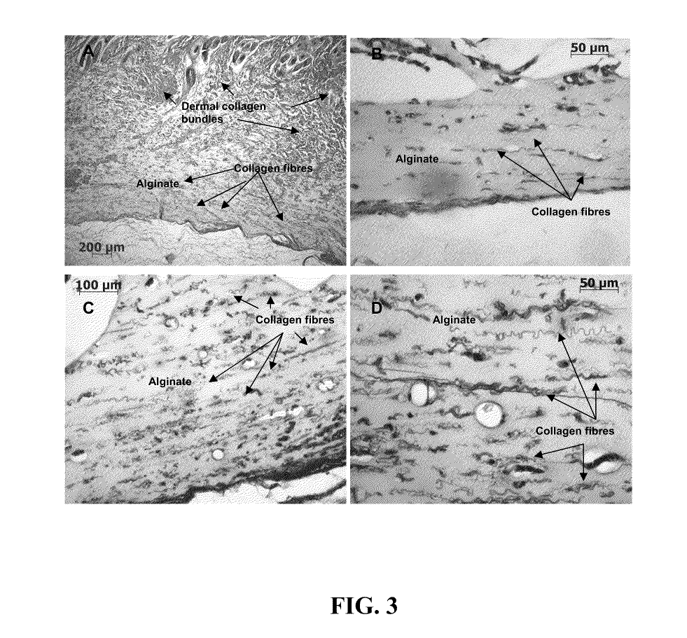

The corresponding van Gieson stains of the removed sites after 4 weeks are documented in FIG. 3 A-D. FIG. 3 shows collagen stains (van Gieson staining) of the injection sites with test implants 1 (A, B) and 2 (C, D) four weeks after injection into the rabbit. In particular, FIG. 3A shows collagen fibres within the i.d. injected test implant; the collagen fibres are uniformly ruby-red in colour, like the original dermal collagen bundles. FIG. 3B shows a detailed photomicrograph of the s.c. implanted test implant 1, which is interspersed with collagen fibres. FIG. 3C shows a s.c. implanted test implant 2, which is interspersed with a large number of collagen fibres, and FIG. 3D describes a detailed photomicrograph of the s.c. implanted test implant 2, which is interspersed with collagen fibres of different lengths.

Accordingly, it has been possible to show that the monolithic alginate implant prepared according to the present invention is not only very compatible with the surroundings, but also promotes the growth of collagen fibrils.

The results after 12 weeks corresponded analogously to those after 4 weeks; that is to say, the monolithic alginate implants are found to be completely intact. In some cases the implant is surrounded by a thin layer of fibroblasts.

Consequently, the alginate solutions injected according to the present invention exhibit rapid endogenous Ca2+ crosslinking, and the monolithic alginate implants that formed are to be found completely intact 4 and 12 weeks after injection. The compatibility of the monolithic alginate implants is in all cases as good as that of the commercial product (reference product, beads); moreover, ingrown collagen fibrils were also clearly visible.

7. Ejection Pressure of (Cosmetic) Fillers

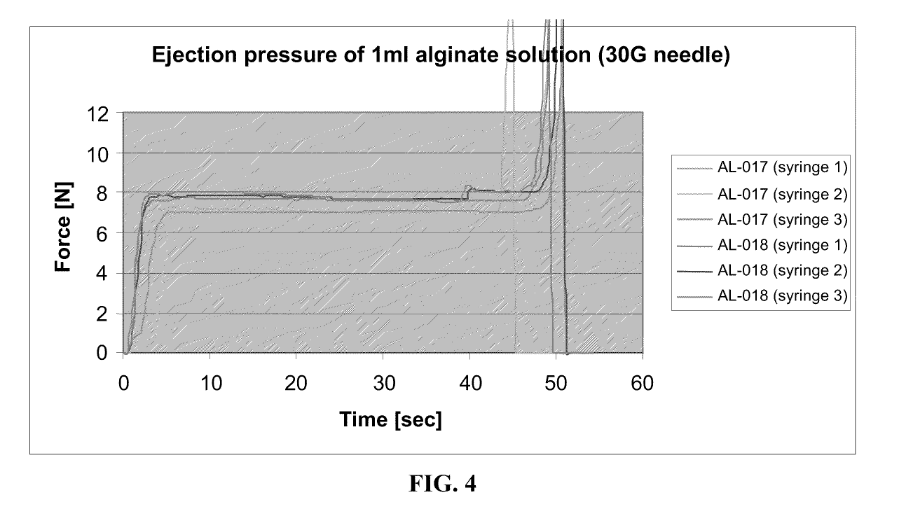

In the following test, the pressure exerted on the syringe was measured in dependence on the product. Various batches of alginate sol, as described under experiment/Example 1, were thereby studied. The necessary pressure was from 7 to 8 newtons in all 6 samples, measured with a 30 G needle (see FIG. 4), that is to say within a readily manageable range.

The ejection pressure of two different, commercially available hyaluronic acid preparations, on the other hand, was in both cases over 25 newtons with a 27 G needle or 30 G needle.

8. Mechanical Stability of Monolithic Alginate Implants

The test arrangement is analogous to Example 3. The aim of the present test was to demonstrate the stability of monolithic alginate implants in dependence on the alginate and in comparison with alginate beads from Example 2.

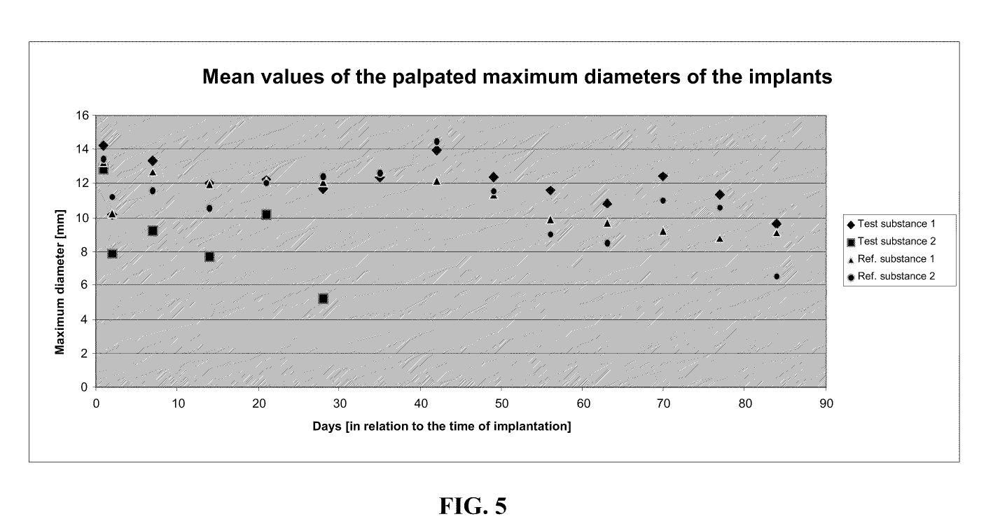

FIG. 5 shows the implant size monitored over 90 days, determined by palpation using a slide gauge. Test substance 1 is a high molecular weight alginate (MW>500,000 g/mol), as is claimed in this invention. Test substance 2 is low molecular weight alginate having a mean MW of less than 120,000 daltons. Reference substances 1 and 2 are alginate beads as described in Example 2 but having a mean diameter of 500 μm.

FIG. 5 shows in this connection the mean values of the palpated maximum and minimum diameters of the various test implants (test substances and reference substances) in the test period. The implant was palpated in the narcotised animal, and the implant size was measured by means of a slide gauge.
Test substance 1 (uncrosslinked alginate solution),
Test substance 2 (low molecular weight uncrosslinked alginate solution),
Reference substance 1 (CellBeads® 500),
Reference substance 2 (CellBeads® 500, autoclaved).

The data are derived from the measurement of the following implant sites: day 1 n=16, day 2-7 in each case n=12, day 14-28 in each case n=8, remaining days in each case n=4.

In summary, this example clearly shows the effect of the in vivo durability of the monolithic alginate implant crosslinked in vivo.

1. A method of making a monolithic alginate implant in a patient, comprising:
providing an uncrosslinked, highly pure and high molecular weight alginate solution, wherein the alginate solution is present in a concentration ranging from 0.5 to 2.5% (w/v) alginate solids content, based on total weight; and
injecting the alginate solution into the patient at a predetermined site to form a gel body comprising the monolithic alginate implant,
wherein the monolithic alginate implant is formed in situ by spontaneous Ca²⁺-crosslinking at the predetermined site without the addition of an exogenous crosslinker.

2. The method of claim 1, wherein the alginate is selected from a highly pure and high molecular weight alginate having a mean molar mass of more than 200,000 daltons.

3. The method of claim 2, wherein the highly pure and high molecular weight alginate comprises at least one of potassium alginate or sodium alginate.

4. The method of claim 1, wherein after injection, monolithic alginate implant bodies form in situ at the injection site.

5. (canceled)

6. The method of claim 3, wherein the alginate of the alginate solution is suspended in a physiological injection solution.

7. The method of claim 1, wherein the alginate solution comprises active ingredients selected from the group consisting of pharmaceutically active compounds, nutrients, marker substances, live cells, and water-soluble auxiliary substances or stabilising agents.

8. The method of claim 6, wherein the pharmaceutically active compounds comprise at least one of vitamins, adhesion proteins, anti-inflammatory substances, antibiotics, analgesics, growth factors, hormones, protein-based active ingredients, peptide-based active ingredients, human growth hormone, bovine growth hormone, porcine growth hormone, growth hormone releasing hormone/peptide, granulocyte-colony stimulating factor, granulocyte macrophage-colony stimulating factor, macrophage-colony stimulating factor, erythropoietin, bone morphogenic protein, interferon or derivatives thereof, insulin or derivatives thereof, atriopeptin-III, monoclonal antibodies, tumour necrosis factor, macrophage activating factor, interleukin, tumour degenerating factor, insulin-like growth factor, epidermal growth factor, tissue plasminogen activator, factor MV, factor IMV, or urokinase.

9. The method of claim 7, wherein the water-soluble auxiliary substances or stabilising agents are selected from the group consisting of proteins, including albumin or gelatin, amino acids, including glycine, alanine, glutamic acid, arginine, lysine or a salt thereof, carbohydrates, including glucose, lactose, xylose, galactose, fructose, maltose, sucrose, dextran, mannitol, sorbitol, trehalose and chondroitin sulphate, inorganic salts, including phosphate, and wetting agents, including TWEEN®, polyethylene glycol, or a mixture thereof.

10. The method of claim 1, wherein the alginate solution further comprises at least one further substance having filler properties.

11. The method of claim 9, wherein at least one further substance having filler properties is present in the alginate solution in amounts by weight ranging from 5 to 50% of the total filler weight (w/w).

12. The method of claim 10, wherein the at least one further substance having filler properties is selected from the group consisting of hyaluronic acid or a salt thereof, collagen, polyacrylamide in a soluble form, cells, plasma proteins and liposuction material.

13. The method of claim 11, wherein the hyaluronic acid is an uncrosslinked, highly purified hyaluronic acid or a salt thereof having a molecular weight in the range from 10,000 as to 10,000,000 Da.

14. The method of claim 11 wherein the salt of hyaluronic acid is selected from sodium hyaluronate, potassium hyaluronate, or ammonium hyaluronate.

15. The method of claim 10, wherein the at least one further substance having filler properties is a solid substance having a particle size ranging from 10 to 150 μm which is insoluble in the alginate solution.

16. The method of claim 14, wherein the solid substance is PMMA microparticles, polylactic acid particles, HEMA particles, or calcium hydroxylapatite particles having a particle form that is substantially round and haying a diameter ranging from 10 to 80 μm.

17. The method of claim 14, wherein the solid substance comprises fibres.

18. The method of claim 16 wherein the fibres are staple fibers and have a diameter ranging from 5 to 40 μm and a length ranging from 20 to 100 μm.

19. The method of claim 16, wherein the fibres consist of tissue-compatible polymers which are biodegradable in the body.

20. The method of claim 16, wherein the fibres consist of collagen, polylactic acid, polylactic acid-glycine copolymers, covalently crosslinked hyaluronic acid, alginic acid, or acrylic and methacrylic acid ester polymers.

21. The method of claim 1, wherein the monolithic alginate implant is dissolvable by injection of a solution comprising at least one of an EDTA solution, a citrate solution, or a complex forming solution.

22. The method of claim 1, wherein the monolithic alginate implant may be used to treat wrinkles, gastrooesophageal reflux disease, urinary incontinence, vesicoureteral reflux disease, tumors, for supporting sphincter musculatures, in reconstructive surgery, or for cosmetic use.

23. A method of using a monolithic alginate implant to treat a medical condition in a patient, comprising:
providing an uncrosslinked, highly pure and high molecular weight alginate solution, wherein the alginate solution is present in a concentration ranging from 0.5 to 2.5% (w/v) alginate solids content, based on total weight; and
injecting the alginate solution into the patient at a predetermined site to form a gel body comprising the monolithic alginate implant,
wherein the monolithic alginate implant is formed in situ by spontaneous Ca²⁺-crosslinking at the predetermined site without the addition of an exogenous crosslinker and
wherein the medical condition requires supporting sphincter musculature, reconstructive surgery, or cosmetic reconstruction.

24. The method claim 22, wherein the medical condition includes at least one of wrinkles, gastrooesophageal reflux disease, urinary incontinence, and vesicoureteral reflux disease.

25. A monolithic alginate implant for use in treating a medical condition, comprising:
a crosslinked, highly pure and high molecular weight alginate gel body,
wherein the implate was formed by injecting into a patient at a predetermined site an uncrosslinked, highly pure and high molecular weight alginate solution, wherein alginate solution is present in a concentration ranging from 0.5 to 2.5% (w/v) alginate solids content, based on total weight, that crosslinked in situ by spontaneous Ca²⁺-crosslinking at the predetermined site without the addition of an exogenous crosslinker.

26.-30. (canceled)
